(19) United States
(12) Patent Application Publication
(10) Pub. No.: US 2023/0104995 A1
(43) Pub. Date: Apr. 6, 2023

(54) SYSTEM AND METHOD FOR BIOLOGICAL AND HYBRID NEURAL NETWORKS COMMUNICATION

(71) Applicant: The Regents of the University of California, Oakland, CA (US)

(72) Inventors: Kateryna Voitiuk, Santa Cruz, CA (US); David H. Haussler, Santa Cruz, CA (US); Mircea Teodorescu, Santa Cruz, CA (US)

(21) Appl. No.: 17/960,093

(22) Filed: Oct. 4, 2022

Related U.S. Application Data
(60) Provisional application No. 63/251,934, filed on Oct. 4, 2021.

(51) Int. Cl. G01N 33/50; G16B 5/00; G16B 40/20

(52) U.S. Cl. CPC G01N 33/5082; G16B 5/00; G16B 40/20

(57) ABSTRACT

An electrophysiological recording and stimulation system includes an electrophysiological device coupled to neural tissue and configured to measure neural electrophysiological signals. The system also includes a computing device coupled to the electrophysiological device and configured to receive neural electrophysiological signals from the electrophysiological device, and transmit neural stimulating signals to the neural tissue through the electrophysiological device based on the neural electrophysiological signals.

### CROSS-REFERENCE TO RELATED APPLICATION

The present application claims the benefit of and priority to U.S. Provisional Application No. 63/251,934, filed on Oct. 4, 2021. The entire contents of the foregoing application are incorporated by reference herein.

### GOVERNMENT LICENSE RIGHTS

This invention was made with government support under Grant No. R01MH120295, T32HG008345 awarded by the National Institutes of Health and National Science Foundation, Grant No. 2034037. The Government has certain rights in the invention.

### BACKGROUND

During initial stages of development, the human brain self assembles from a vast network of billions of neurons into a system capable of sophisticated cognitive behaviors. The human brain maintains these capabilities over a lifetime of homeostasis. While progress has been made studying large-scale brain patterns or behaviors, as well as understanding the brain at a cellular level, it remains unclear how neural interactions create meaningful cognition. This missing link—a theory that would unify processes at the macroscopic and microscopic scale—is the most intriguing problem in modern neuroscience.

The pace of progress in neuroscience depends on experimental toolkits available to researchers. New tools are required to explore new forms of experiments and to achieve better statistical certainty. Recently, brain organoids, three-dimensional neural tissue structures generated from human stem cells, have entered the neuroscience toolkit, enabling researchers to model neural development and connectivity. Electrophysiological experiments in cerebral organoids have shown neural activity. Organoids are more realistic than two-dimensional cultures, recapitulating the brain, which is inherently three-dimensional.

However, organoids receive no structured neural inputs from an outside environment. Thus, there is a need to address this limitation to further increase usefulness of organoids in neurological experimental studies.

### SUMMARY

Extracellular voltage recordings from in vitro cell cultures allow for investigation of neural activity and dynamics. In particular, these recordings allow for assessing information processing in complex neuronal networks and enable discovery on a scale from single neuron firing patterns to local and long-range functional connectivity, network synchrony, and oscillatory activity.

The present disclosure merges biology with software to accelerate the pace of neuroscience discoveries and is centered around a new type of organoid, a hybrid organoid. As used herein, the term “organoid” denotes a three-dimensional neural tissue structure generated from human stem cells, an “assembloid” is an assembly of fused organoids of different tissue types, and a “connectoid” is an assembly of organoids connected by axon bundles. A hybrid connectoid according to the present disclosure is a connectoid in which the component organoids are also connected to each other in silico, in addition to or alternatively to the axon bundles. As used herein, the term “in silico” denotes a connection of each of the organoids of the connectoid to a computing device, such as an electrophysiological recording and stimulation system.

The present disclosure provides an inexpensive electrophysiological recording system that is easily accessed and controlled via a standard web interface through IoT protocols. The system may include any suitable computing device, which in embodiments may be a low cost, single-board computer (SBC), such as, Raspberry Pi. The computing device acts as the primary processing device and is configured to operate with a hardware expansion circuit board and software, which provides voltage sampling and user interaction.

The system according to the present disclosure provides an all-in-one electrophysiology and processing system that can simultaneously record data from multiple channels in the mV scale. The disclosed system may be used with a wide range of electrode probes including, but not limited to, rigid 2D and flexible 3D microelectrode arrays (MEAs), silicon probes, and tetrodes. The system may also be used in long-term experiments with full automation using programs that can optimize experimental variables. This disclosure also provides examples validating the system's accuracy and reliability for measuring neural activity.

The system records signals from neural tissue using a versatile circuit board connecting to neurorecording (electrophysiology amplifier) chips (e.g., Intan RHD series) to perform highly sensitive analog-to-digital (A/D) conversion. Data from the chips may be optionally preprocessed on-site using the SBC computer and streamed to a cloud service where further sorting and analysis of detected spikes may be performed. Spike sorting analysis may be used to measure neural activity changes over time in individual neurons and networks of neurons, using features such as spike waveform, frequency of activity, and correlation to the activity of nearby neurons.

In one embodiment, an electrophysiological recording and stimulation system is disclosed. The system includes an electrophysiological device coupled to neural tissue and configured to measure neural electrophysiological signals. The system also includes a computing device coupled to the electrophysiological device and configured to receive neural electrophysiological signals from the electrophysiological device and transmit neural stimulating signals to the neural tissue through the electrophysiological device based on the neural electrophysiological signals.

Implementations of the above embodiment may include one or more of the following features. According to one aspect of the above embodiment, the computing device may be further configured to execute software instructions embodying a simulated neural network. The computing device may be also configured to provide neural communication between the simulated neural network and the neural tissue. The simulated neural network may be a reinforcement learning (RL) agent. The system may further include a monitor configured to display a graphical user interface having a graphical representation of the simulated neural network and the neural tissue. The electrophysiological device may be a microelectrode array having a plurality of electrophysiological electrodes. The neural tissue may include at least one organoid. The electrophysiological recording and stimulation system may further include a scaffold disposed on the microelectrode array. The scaffold may be configured to hold a plurality of organoids in a plurality of wells. Each organoid may be connected to a plurality of neighboring organoids of the plurality of organoids.

In another embodiment, a method for electrophysiological recording and stimulation is disclosed. The method includes measuring neural electrophysiological signals of neural tissue through an electrophysiological device. The method also includes receiving, at a computing device, neural electrophysiological signals from the electrophysiological device. The method further includes transmitting neural stimulating signals to the neural tissue through the electrophysiological device based on instructions from the computing device.

Implementations of the above embodiment may include one or more of the following features. According to one aspect of the above embodiment, the method may also include executing software instructions embodying a simulated neural network. The method may further include providing neural communication between the simulated neural network and the at least one organoid through the computing device. The simulated neural network may be a reinforcement learning agent. The method may additionally include displaying a graphical user interface having a graphical representation of the simulated neural network and the neural tissue. The electrophysiological device may be a microelectrode array having a plurality of electrophysiological electrodes. The neural tissue may include at least one organoid. The electrophysiological device may include a scaffold disposed on the microelectrode array. The scaffold may be configured to hold a plurality of organoids in a plurality of wells. Each organoid may be connected to a plurality of neighboring organoids of the plurality of organoids.

### BRIEF DESCRIPTION OF DRAWINGS

Various embodiments of the present disclosure are described herein below with reference to the figures wherein:

FIG. 1 are schematic diagrams of different types of organoids including a hybrid connectoid according to an embodiment of the present disclosure;

FIG. 2 is a schematic diagram of a hybrid connectoid according to an embodiment of the present disclosure;

FIG. 3 is a schematic diagram of a hybrid connectoid according to another embodiment of the present disclosure;

FIG. 4 are photographs of scaffolds including the hybrid connectoids of FIGS. 2 and 3 according to the present disclosure;

FIG. 5 are enlarged photographs of the hybrid connectoid of FIG. 2;

FIG. 6 is a perspective view of an electrophysiological recording and stimulation system according to an embodiment of the present disclosure;

FIG. 7 is a schematic, top view of an interface board assembly according to an embodiment of the present disclosure;

FIG. 8 is a schematic architecture view of the interface board according to an embodiment of the present disclosure;

FIG. 9 is a schematic diagram of an electrophysiological recording and stimulation system according to another embodiment of the present disclosure;

FIG. 10 is a schematic diagram of an interface device and a multi-well microelectrode array;

FIG. 11 are graphical representations of connections between organoids according to an embodiment of the present disclosure;

FIG. 12 is a schematic diagram of the electrophysiological recording and stimulation system of FIG. 10 connected to the hybrid connectoid of FIG. 2 according to an embodiment of the present disclosure;

FIG. 13 is a schematic diagram of a reinforcement learning agent according to an embodiment of the present disclosure; and

FIG. 14 is a system for inducing learning from structured inputs in organoids of the hybrid connectoid according to an embodiment of the present disclosure.

### DETAILED DESCRIPTION

The present disclosure provides for a system and method for novel interfaces with neural tissue, such as brain organoids of a connectoid. The connectoid, including its plurality of interconnected organoids are placed in a scaffold, which is then electrically coupled to a neurophysiological recording system. The system provides an alternative channel of communicating between connected organoids of the connectoid allowing for recordation of neurological signals and transmission of stimulated neurological signals to monitor and study the organoids.

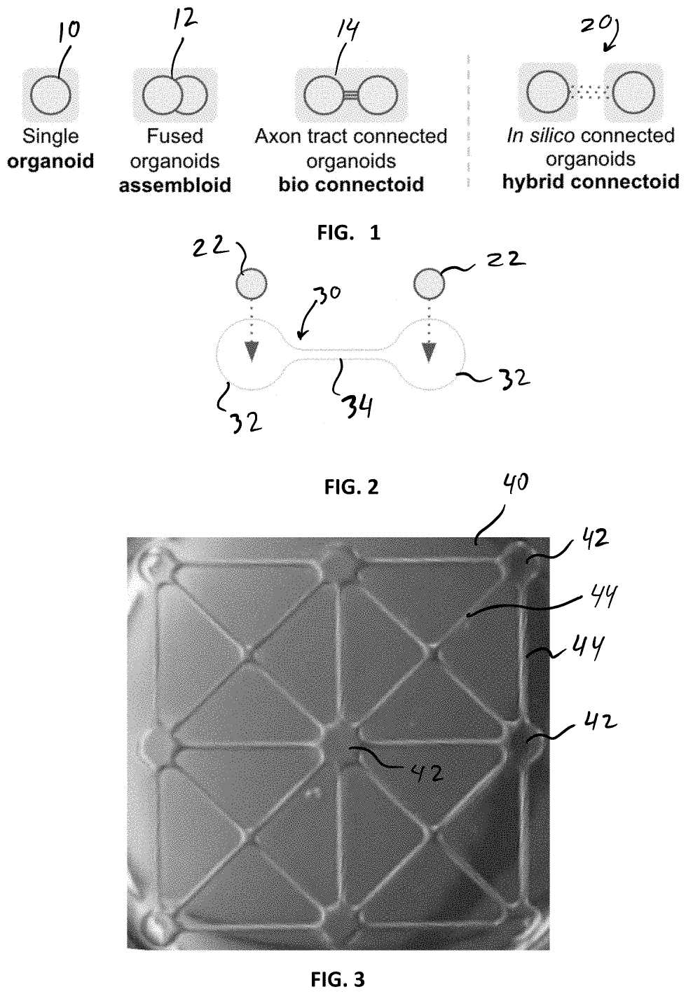

With reference to FIG. 1, different types of organoids are shown, which include a single organoid 10, an assembloid 12, which includes a plurality of fused organoids, and a connectoid 14, which includes an axon tract connecting a pair of organoids. With reference to FIGS. 1 and 2, a hybrid connectoid 20, which in addition to providing a physical channel for an axon bundle also provides an electrical, i.e., computing hardware, connection between two or more organoids 22. The connectoid 20 may be disposed in a scaffold 30 having a plurality of wells 32 interconnected by a channel 34 (FIG. 2). Each organoid 22 is placed within a well 32 allowing for the axon bundles 24 to form between the organoids 22 through the channel 34 allowing for the organoids 22 to connect to each other.

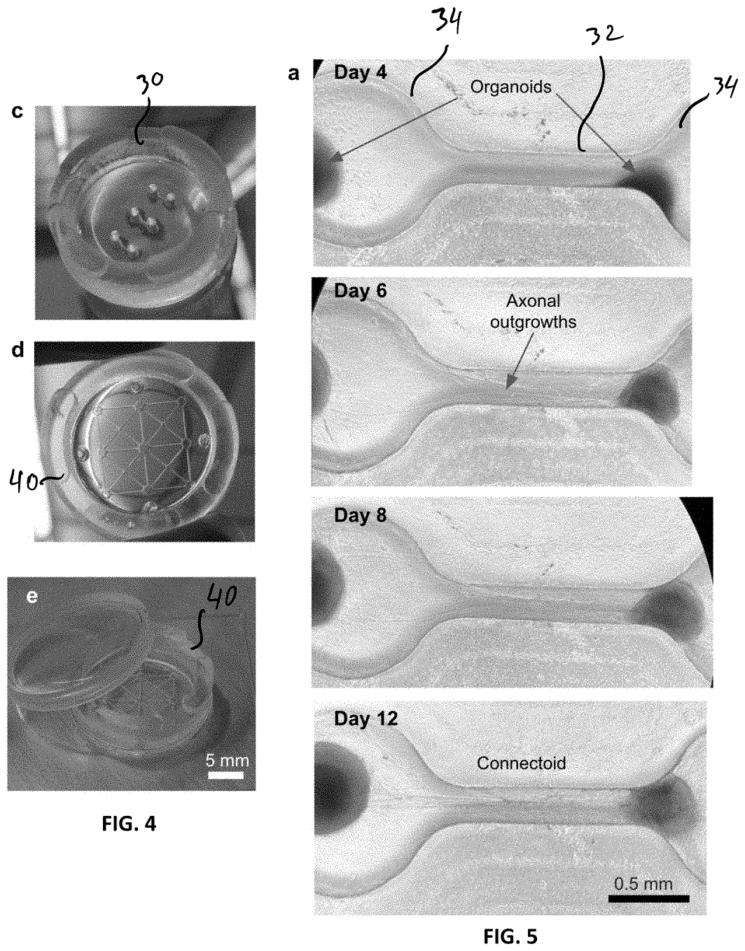

With reference to FIGS. 2 and 4, the scaffold 30 may be formed from any suitable nonconductive polymer, such as silicon-based polymer, such as polydimethylsiloxane (PDMS), using any desirable manufacturing process, such as spin coating. In embodiments, the wells 32 of the scaffold 30 may have a cylindrical shape having a diameter from about 1 mm to about 3 mm and the channel 34 may have a length of from about 2 mm to about 5 mm and a width from about 0.1 mm to about 0.5 mm.

With reference to FIG. 3, a scaffold 40 according to another embodiment of the present disclosure may include a plurality of wells 42 allowing for several (e.g., more than 3) organoids 22 to link together to form the hybrid connectoid 20. As shown in FIG. 3, the wells 42 may be arrange in a two-dimensional matrix, e.g., 3×3, with each of the wells 42 interconnected to a plurality of neighboring wells 42 by a corresponding channel 44. In certain embodiments, the channels 44 may intersect with each other. This intersecting design allows several organoids 22 to connect to each other.

The organoids 22 may be artificially grown in vitro for any suitable period, e.g., 5 weeks, and are then placed in the wells 32 or 42 of the scaffolds 30 or 40, respectively. As shown in FIG. 5, after approximately 12 days of being placed in the scaffold 30, two organoids 22 develop axonal growths that eventually developed into the axon bundles 24 and form the hybrid connectoid 20. Hybrid connectoids 20 according to the present disclosure may also be used to link more than two organoids 22 since the hybrid connectoids 20 may also be connected electronically via a computing device, which enables a flexible new level of organization and control for brain modeling experiments and allows for interfacing machine and biological learning directly.

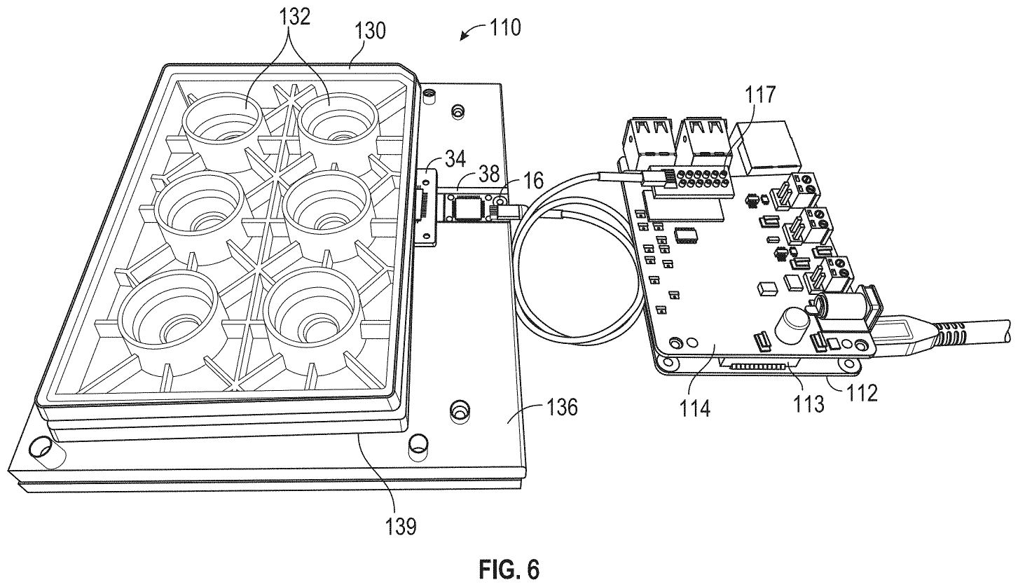

FIG. 6 shows an electrophysiological recording and stimulation system 110 configured for closed-loop neural monitoring and stimulation of organoids 22. The system 110 provides a flexible, programmable platform to record and perturb in vitro cultures, such as organoids 22, in a closed-loop manner to measure properties of neural tissue in response to stimuli over time. Many current platforms that allow closed-loop stimulation were not designed for speed, having slow response rates (e.g., longer than 100 ms) due to interface protocol delays and overhead of user abstractions to protect proprietary designs. In addition, most electrophysiology platforms require a dedicated personal computer and are generally expensive (e.g., tens of thousands of dollars). Combined, these factors make experiments difficult for many organoids and experimental replicates at the required performance. The system 110 provides response times that are 10 ms or faster while being much more cost effective.

Thus, the system 110 provides an inexpensive optimized electronics platform for interfacing with biological neural models. The system 110 is also built for long-term experiments with automation using programs that can optimize experimental variables. As described in further detail below, the architecture of the system 110 incorporates hardware expansion boards that enable the use of low cost, single-board computers (SBC), such as, Raspberry Pi, to interface with bioamplifier chips (e.g., Intan REID series.)

The system 110 includes a computing device 112 coupled to an interface device 114. The computing device 112 may be any suitable computing device such as, SBC, which provides a low cost, miniature computing platform. In embodiments, the computing device 112 may be a Raspberry Pi, e.g., Model 3 B+, which is a low-cost, small-scale, SBC with a quad-core ARM Cortex-A53 processor, an input/output system memory, and storage, including expandable storage for use with removable flash card. Raspberry Pi may also be programmed to interface with customized hardware with a standard data communication protocol.

The interface device 114 is coupled to the computing device 112 via a header connector 113. The interface device 114 is also coupled to an electrophysiology amplifier chip 116, which may be an Intan RHD2132 electrophysiology amplifier chip. The interface device 114 includes an electrophysiological chip adapter 117 that is coupled to the amplifier chip 116. The electrophysiology amplifier chip 116 amplifies voltage signals sensed by the electrodes and converts the analog signals to digital values for storage and buffering by the computing device 112. The amplifier chip 116 may have any number of communication channels, which may be from 16-400. Thus in an embodiment of 400 channels, 16 organoids may be observed with 25 channels per individual organoid.

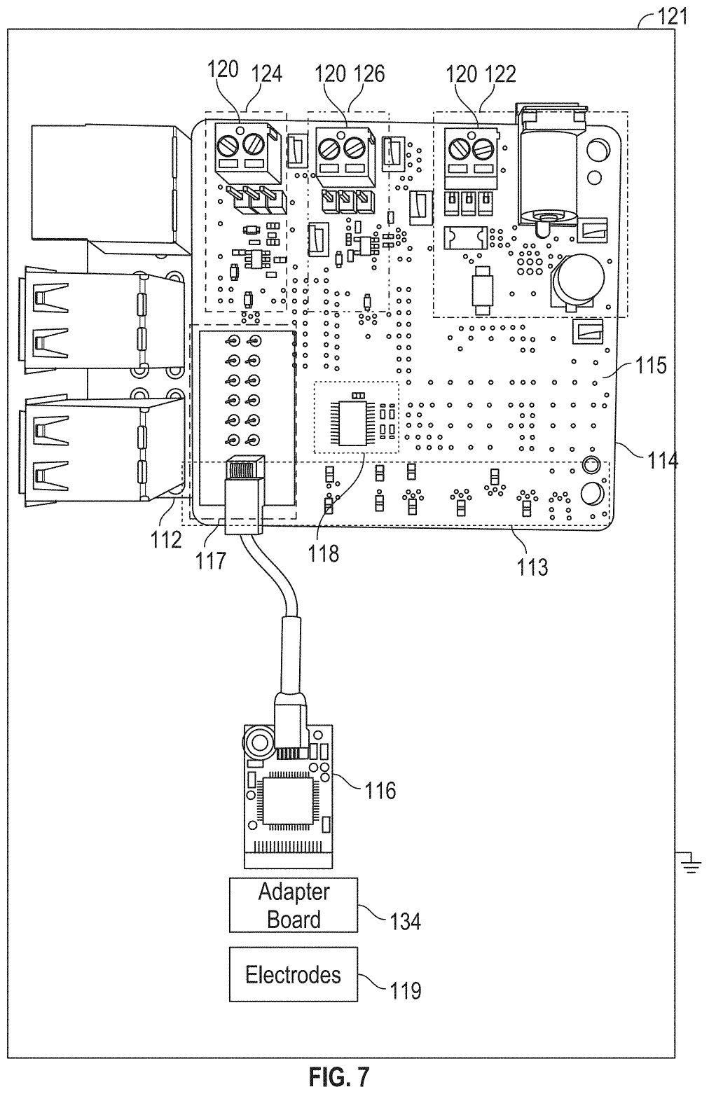

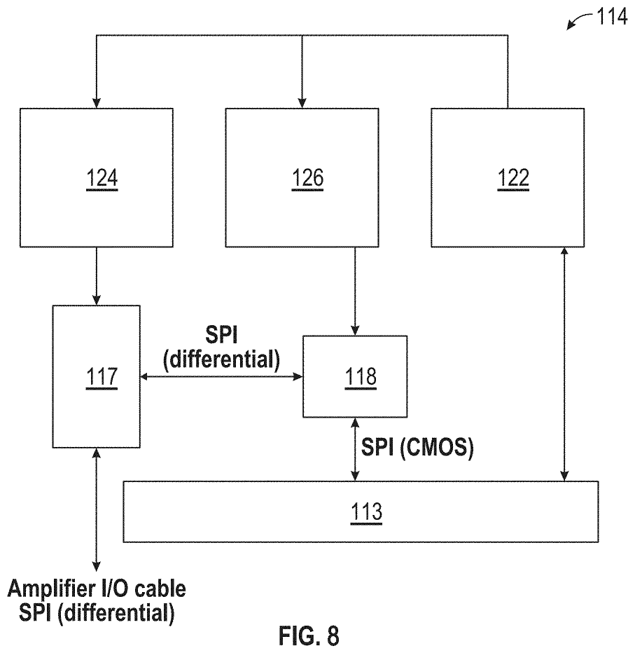

With reference to FIGS. 7 and 8, the interface device 114 enables communication between the amplifier chip 116 and the computing device 112. The amplifier chip 116 is configured to use low-voltage differential signaling (LVDS) to reduce the effects of noise and electromagnetic interference (EMI) and allow increased cable length. However, the computing device 112 is configured to communicate using complementary metal-oxide-semiconductor (CMOS) level logic. To translate between the two signal types, the interface device 114 includes an LVDS converter 118. The LVDS converter 118 includes four LVDS line drivers and one LVDS line receiver to control data lines for communicating with the amplifier chip 116 over its serial peripheral interface (SPI). The LVDS converter 118 is coupled to the header connector 113 and to the electrophysiological chip adapter 117 allowing the LVDS converter 118 to convert signals between the computing device 112 and the amplifier chip 116.

Communication between the computing device 112 and the amplifier chip 116 may use serial peripheral interface (SPI), which provides a fast and synchronous interface that is widely used in embedded systems for short-distance data streaming. SPI is a full-duplex leader-follower-based interface allowing leader and follower devices to transmit data at the same time.

The protocol for the computing device 112 and the amplifier chip 116 may be a four-wire (i.e., four-channel) interface including the following signals: clock (SCLK), chip select (CS), leader-out-follower-in (LOFI), and leader-in-follower-out (LIFO). In particular, the computing device 112 is configured to communicate with the LVDS converter 118 over a four-channel interface, with the LVDS 118 communicating with the amplifier chip 116 over the SPI. The computing device 112 acts as the leader device and generates a clock signal and transmits the same through SCLK. The computing device 112 also outputs recording commands to configure the amplifier chip 116 through LOFI. The amplifier chip 116 responds as follower and sends the digitized data back by LIFO. The amplifier chip 116 allows configuration of sampling rate and bandwidth of the low-noise amplifiers. Each of the channels on the amplifier chip 116 may be sampled sequentially with available sampling rate from about 2 kHz to about 115 kHz per channel. The amplifier chip 116 may provide about 46 dB midband gain with lower bandwidth from 0.1 Hz to 500 Hz and upper bandwidth from 100 Hz to 20 kHz.

Besides translation between signal types, the interface device 114 also provides different levels of power derived from a power source input 120, which may be about +5V. The single source input powers the computing device 112 and the interface device 114 and may be supplied either through a power connector 122 of the interface device 114 or through a power connector (e.g., micro-USB) of the computing device 112. The power source input 120 may be coupled to the header connector 113 powering the computing device 112 therethrough. The power connector 122 may include high-frequency power line noise filter, e.g., ferrite beads, to remove high-frequency power line noise. The interface device 114 is also configured to convert input power to voltage levels suitable for powering the amplifier chip 116 and the LVDS converter 118. In particular, the input power may be converted to an amplifier power input 124, e.g., +3.5V, for the amplifier chip 116 and a converter input 126, e.g., +3.3V, for the LVDS converter 118. Conversion may be performed by low-noise linear voltage regulators to smooth and isolate any fluctuations from the power supply.

The interface device 114 includes a printed circuit board (PCB) 115 with each of the components (e.g., LVDS converter 118, header connector 113, etc.) disposed thereon. The PCB 115 includes four conductive layers (e.g., copper) with the top and bottom layers of the board being grounded, while two inner layers providing for transmission of signal and power, respectively. Every via of the signal layer has a ground via next to it to sink electromagnetic interference (EMI) as signals switch layers. Via stitching may be done around the perimeter of the PCB 115 and throughout the board area to separate components of the interface device 114 and fill in areas with no components. The amplifier chip 116 and the computing device 112 are separated by a cable such that noise from the computing device 112 would not interfere with the sensitive neural signal recording. The interface device 114 may also include an additional controller, e.g., CPU or FPGA, to increase sampling rate and precision of timing in between samples.

The amplifier chip 116 is configured to connect to a plurality of electrophysiological electrodes 119. In embodiments, an electrophysiological device, such as a multi-well microelectrode array (MEA) 130 may be coupled to the amplifier chip 116. In embodiments, any other suitable neural electrode device may be used. The MEA 130 may include a plurality of wells 132, e.g., 6-well MEA plate from Axion Biosystems, each of which includes one or more electrodes 119 that are coupled to the amplifier chip 116. The MEA 130 is disposed over an adapter board 134 with the contacts of the MEA 130 engaging contacts, e.g., spring finger pins, of the adapter board 134. The adapter board 134 is disposed in a board housing 136 defining a first cutout 138 for the adapter board 134 and a second cutout 139 for the MEA 130. The first and second cutouts 138 and 139 of the board housing 136 align MEA 130 with the adapter board 134 ensuring consistent mating of spring finger pins to electrode contacts. The board housing 136 may include a plastic interior surrounded by aluminum plates and compressed together by fasteners or any other suitable method, e.g., adhesive. The aluminum plate prevents the warping of the plastic and ensures even pressure compressing the plate and connector on both sides.

In embodiments, during data acquisition, the system 110 may be shielded by a Faraday cage 121. The Faraday cage 121 is configured to block electromagnetic fields in order to reduce environmental noise and maximize the signal-to-noise ratio (SNR) during electrophysiological signal recording. The Faraday cage 121 may be a rectangular box made of 1 mm thick steel sheets with a power line connected to an earth ground. A 60 Hz infinite impulse response notch filter may be used to remove the power line noise before recording electrophysiological signals. In addition, a 300-6000 Hz 3rd order Butterworth bandpass filter may also be used to attenuate frequency components outside the neural activity range.

Signal-to-noise ratio may also be improved with enabling and tuning on-chip filtering and improving Faraday cage shielding. In vitro cultures typically fire with amplitudes between 10-40 mV, and require sensitive recording equipment, as an increase of just a few mV in noise for spikes on the lower end of the spectrum would be a non-trivial variable.

The present disclosure also provides a system and method enabling a cloud-based experiment platform in which biological measurement and local computing and sensing hardware are presented to the user through the cloud, such that experiment management and control can be administrated remotely and may be automated by a computer application. Biological, i.e., neural, recording is performed by local hardware, which then transmits the collected data to a cloud, i.e., one or more servers, that is accessible by a user. The cloud provides the user with access to the local hardware as well as the collected data.

The system 110 is configured to perform biological sampling as well as to record and store physiological data. The computing device 112 is configured to run software that communicates with the amplifier chip 116 and stores the digitized electrophysiological signals as data.

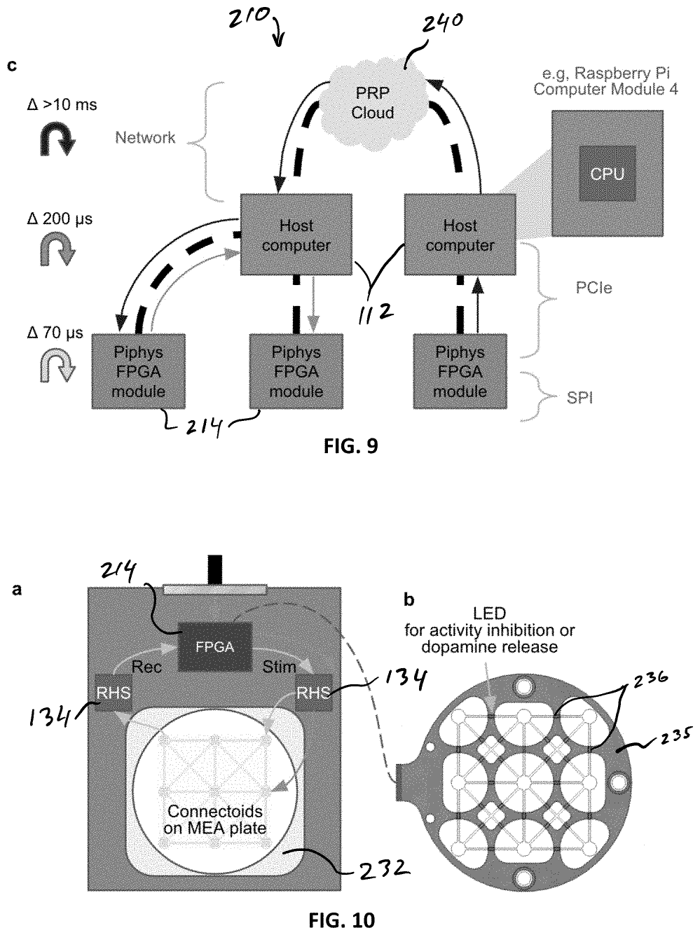

FIG. 9 shows an electrophysiological recording and stimulation system 210 according to another embodiment of the present disclosure. The system 210 is similar to the system 110 and the differences therebetween are described below. The system 210 is configured to interface with the MEA 130, and includes an interface device 214, having an FPGA configured to maintain a steady sampling rate and buffer data for the computing device 112, solving timing issues that may be introduced by an operating system (OS) scheduler of the computing device 112. The interface device 214 also allows for pre-processing signals and prototyping of on-chip spike sorting algorithms useful in neuroengineering.

As shown in FIG. 10, the interface device 214 is coupled to the adapter board 134, which is in turn coupled to a 9-well MEA 230. The MEA 230 includes a mounting plate 232, which may be formed from glass, configured to receive the scaffold 30 or 40, which may be adhered thereto using adhesive or adhesive properties of the scaffold 30 or 40. In particular, the scaffold 40 of FIG. 4 is shown being disposed in the MEA 230 in FIG. 9. The MEA 230 includes a 9-electrode array which aligns with the wells 42 of the scaffold 40 once the scaffold 40 is disposed within the MEA 230.

In further embodiments, an optogenetics accessory 235 may also be attached to the MEA 230. The optogenetics accessory 235 includes a plurality of light emitting devices (LEDs) 236 configured to illuminate the axon bundles 24 interconnecting the organoids 22. In particular, the LEDs 236 are aligned to be disposed underneath the channels 44. The optogenetics accessory 235 is also controlled by the interface device 214 and may be used to stimulate genetically engineered light-sensitive neurons of the organoids 22, e.g., inhibit activity, release dopamine, etc. by illuminating the axon bundles 24 disposed in the channels 44.

With reference to FIG. 9, the system 210 may include a plurality of interface devices 214. Each computing device 112 may be coupled to one or more of the interface devices 214 using peripheral component interconnect express (PCIe) port or any other suitable communication interface. Each of the interface devices 214 is coupled to an MEA 230 using SPI ports or an any suitable connection. In addition, each of the computing device 112 is also in communication with a cloud computing platform 240 and transmits the data to the platform 240 for permanent storage and access by the user. The platform 240 may be a remote server, a cloud server or service, e.g., Amazon Web Services (AWS) Simple Storage Service (S3), or any other computing platform. The platform 240 may also execute various applications. As used herein, the term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, a personal computer, or a server system.

The computing device 112 may be coupled to a communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

With continued reference to FIG. 10, path delays of round-trip signal transmission times for each communication level of the system 210 may be as follows, e.g., between the interface device 214 and the MEA 230 being about 70 μs, between the interface device 214 and the computing device 112 being about 200 μs, and between the computing device 112 and the platform 240 being about 10 ms. Thus, the interface device 214 supports inter-organoid communication and feedback through three pathways: within the interface device 214, the computing device 112, and the platform 240.

The system 210 provides automated electrophysiology infrastructure, which enables the creation of a live feedback loop between a computer application and an active neural circuit suitable for exploring local learning phenomena such as Hebbian and anti-Hebbian synaptic changes, spike timing-dependent plasticity (STDP) and other neuroscience learning models with instructive pathway input. The amplifier chip 116 provides recording and stimulation capabilities, allowing the system 210 to record from and excite neurons in submillisecond time frames, sufficient for most hypothesized learning mechanisms.

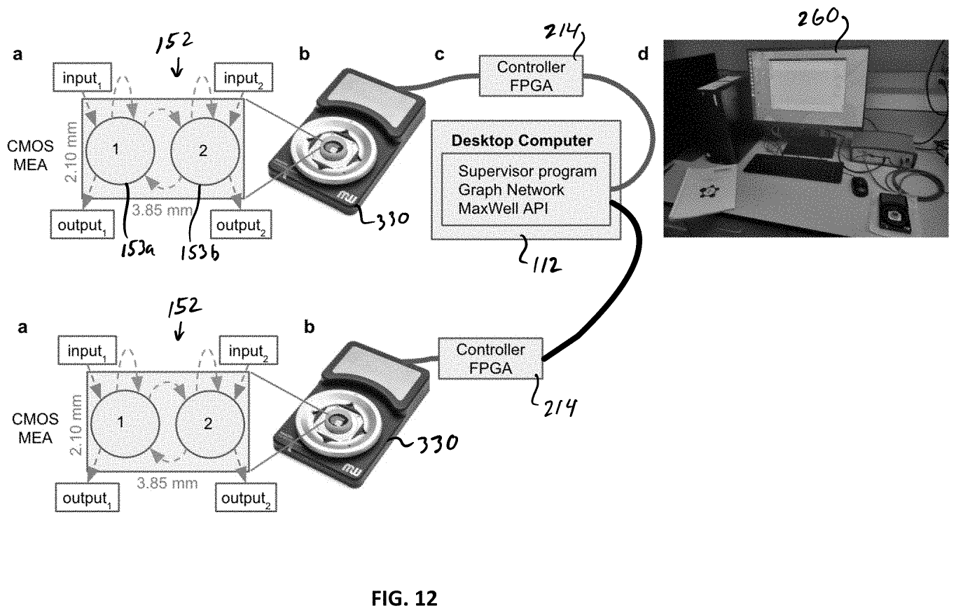

Single organoids may be limited in size due to necrosis, while assembloids and connectoids are limited by the spatial constraints of a culture dish. Hybrid connectoids 20 according to the present disclosure are linked in silico through the system 210, which allows for connecting multiple organoids 22 across multiple culture dishes (e.g., FIG. 12 with different scaffold 30 or 40 disposed in individual MEA 230) but also for connecting organoids 22 with entirely simulated models of the brain or artificial neural networks. The simulated neural models may be executed as software applications by the computing device 112 and/or the platform 240. Hybrid connectoids 20 linked in silico are easily accessible digitally, which expedites computer-driven computer-optimized experiments. The following sections describe how in silico connections are formed and the flow of information between in silico linked hybrid connectoids 20.

A neural circuit, connectoids, organoids, or large-scale brain network may be represented as graphical representations of entities and connections between them. Each entity may be a single neuron, groupings of neurons, brain regions, sensing organs, or actuators, or simulations of any of these. Connections may be single synapses, sets of synapses on single axons, or sets of synapses on bundles of axons from sets of neurons, or simulations of these. This graphical representation may function on different levels of hierarchy and abstraction, similar to the structural hierarchy found in brains. The graphical representation network may also be software-defined and accessible by users to construct, view, and/or modify. The graphical representation may also provide a layer of abstraction to connect multiple neural models within one framework. The choices for neural models may be tool kits such as spiking neural networks in software or on neuromorphic chips, Brain Modeling Toolkit (BMTK), which is a python-based software package for building, simulating and analyzing large-scale neural network models, and other software simulations of neural tissue. The graphical representation may be supported by underlying software mechanisms and hardware infrastructure that fulfills the exchange of signals between neural models in real-time.

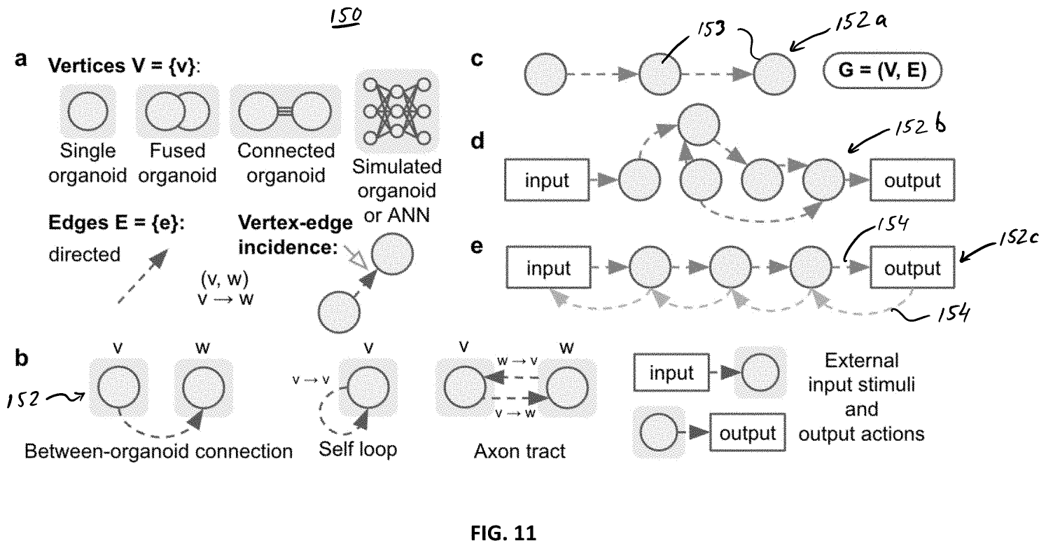

In embodiments, a graphical user interface (GUI) 150 including graphical representations 152 (FIG. 11) may be displayed on a monitor 260 (FIG. 12) coupled to the computing device 112 or the platform 240. The GUI provides a simple language (e.g., block programming) to define communication between biological and/or software neural models, and in doing so, allow users to focus on testing their neuroscience ideas without worrying about logistics.

Exemplary embodiments of the GUI 150 and the graphical representations 152 are shown in FIG. 11. In particular, the graphical representations 152 may be graphs having different types of nodes 153, e.g., single organoid, fused organoid, connected organoid, simulated organoid, an artificial neural network (ANN). In addition to nodes and vertices, the graphical representations 152 also include topological connections shown as edges 154, such as connections between organoids, self-loop, axon tract, external stimuli and output actions. Graphical representations 152 may be in the form of linear graph 152a, an acyclic graph 152b, and/or cyclic graphs 152c, with different types of edges 154 shown between the nodes 153. The graphical representation 152 may be a directed graph of the hybrid connectoid 20 where the nodes 153 are neurons (or population of neurons), and an edge 154 is a set of synapses. The graphical representation 152 is at least weakly connected, that is, there is at least one undirected path between any pair of nodes 153. Nodes and edges may also implement functions to modify signals. Node functions, ƒ, represent transformations of inputs by the biological neural network (e.g., organoids 22) and edge functions, f, are transformations of outputs applied by the computing device 112 during transmission along the digital channel. Generally, the graphical representations 152 may be used to connect various types of biological tissues and artificial neural models.

With reference to FIG. 12, the system 210 may also be connected to one or more high-density single well MEA 330, which may be MaxOne Single-Well MEA available from MaxWell Biosystems. The MEA 330 may include a plurality of electrodes, e.g., 26,400 (9.3×5.45 sq-μm, 17.5 μm pitch) and may have about 1,204 readout channels with 32 simultaneous channels. The MEA 330 is connected to the interface device 214 and a computing device 212, which may be a desktop computer or the computing device 212, that is also coupled to the monitor 260. The computing device 212 is configured to run any suitable application for communicating and controlling the interface device 214 and the MEA 330. The hybrid connectoid 20 is disposed inside the scaffold 30, which is in turn, placed in the MEA 330. The system 210 interfaces with the hybrid connectoid 20 through the GUI 150, which includes a graphical representation 152 that is displayed on the monitor 260. The graphical representation 152 includes two nodes 153a and 153b representative of the two organoids 22 of the hybrid connectoid 20. The computing device 212 and/or the interface device 214 are configured to perform the edge function calculations and activation functions. The computing device 212 also sends commands to stimulate electrodes of the MEA 330 according to the data transmissions through the graphical representation 152.

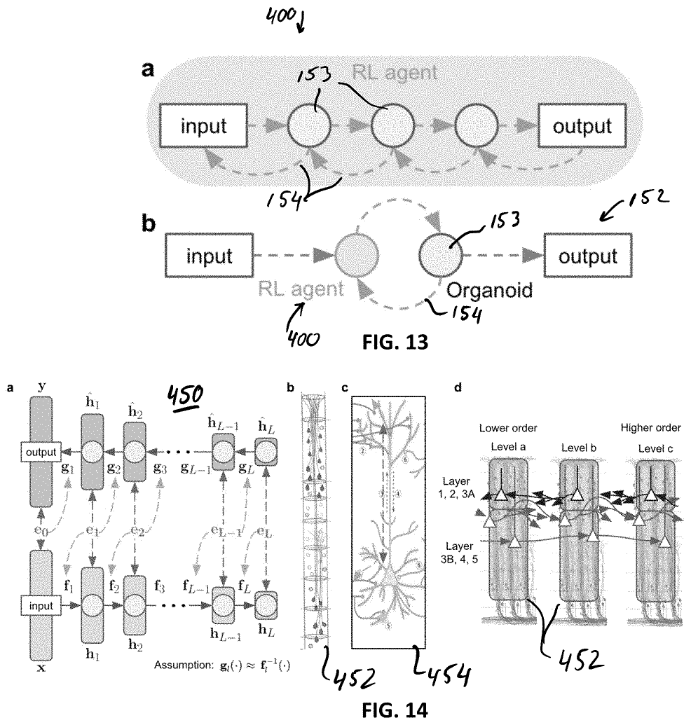

FIG. 13 shows a machine learning (ML) algorithm 400, such as reinforcement learning agent, configured to utilize the graphical representation 152 to interact with organoids 22 through the MEA 330 and/or neural simulations executed by the computing device 220. The ML algorithm 400 is implemented as software executable by computing device 220.

The GUI 150 and the framework of the graphical representation 152 allows setting up reinforcement learning experiments by structuring the problem (i.e., providing clear I/O interfaces and edge functions for modification). The RL agent observes signals from electrodes of the MEA 330 coupled to the organoids 22 and changes electrode functions and mappings to conduct meta-learning. Thus, the computing device 212 is configured to monitor neural activity of the organoids 22 (e.g., receive electrode signals) but also to control the MEA 330 to output electrical signals through the electrodes to simulate neural activity in the organoids.

The ML algorithm 400 is configured to interact with the graphical representation 152 in two different configurations. First, the ML algorithm 400 is configured to monitor the input and output of the graphical representation 152, communications between nodes 153, and can modify edge functions f of the graphical representation 152 to drive the graphical representation 152 towards the desired target output. Second, the ML algorithm 400 may be embedded within the graphical representation 152 as a node 153 and can send discrete signals to the organoid 22 or set of organoids 22 to guide them towards a more appropriate response. The ML algorithm 400 may be trained on a dataset of neural signals collected by the computing device 212.

The ML algorithm 400 may be used to implement any biologically plausible learning algorithms, such as target propagation, learning in spiking neural networks, and learning in predictive coding. The ML algorithm 400 may also be used rapidly assess how biological neural networks learn online by applying computer-mediated stimulus-response reinforcement through a feedback loop between perturbations, recording, and analysis. The framework of the ML algorithm may also be based on empirical studies of biological brains and theoretical models of biologically inspired learning algorithms.

The ML algorithm 400 may also be used to assay feedforward-feedback rhythms and target propagation in organoid-based cultures. As described above with respect to FIG. 12, the system 210 may be used to establish remote communication between physically distanced tissues representing different brain regions mediated and observed by the computing device 212. Thus, two or more hybrid connectoids 20 may be used to represent different brain regions, each of which is connected via their respective MEAs 330 to the computing device 212. The organoids 22 are connected through electrodes of the MEA 330, creating signal propagation layers, using the hybrid connectoid graphical representation 152. This setup allows for analyzing and manipulating the propagation of the signal through neural network layers in vitro.

In the human cerebral cortex, convolutional neural networks are structured in areas of high local connectivity with long-range sparse connections between areas. There is also evidence for feedforward and feedback circuits that alter feedforward activity in the brain. Human cortical organoids contain developing layers and columns similar to those found in the human cerebral cortex. Thus, the system 210 may be used to emulate feedforward and feedback activity in vitro, long-range signals are propagated between individual organoids with high local connectivity. With reference to FIG. 14, the system 210 may accomplish this by: (1) providing an input vector to the feedforward circuit, (2) measuring circuit-wide activity including the output of the feedforward circuit, (3) externally creating a reinforcement vector based on this output, (4) providing the reinforcement vector to the paired feedback circuit associated with the output of the organoid, and (5) again measuring circuit-wide activity. Thus, feedforward and feedback connections between organoids are created where the output of one organoid is passed as the feedforward input into the next organoid through electrodes.

Reinforcement stimuli are passed back as transformations of an organoid's output to the previous organoid. This architecture simulates a multi-layer target propagation. In particular, the reinforcement vector may simulate reward prediction error (RPE) where signals from midbrain dopamine neurons encode the discrepancy between the expected reward (i.e., the feedforward output) and the actual reward (i.e., externally defined training value). Iterating this learning cycle, results in the organoid learning while being trained by an externally defined series of inputs and reinforcements based on its outputs.

Reinforcement may also be used to confirm other forms of non-Hebbian learning that utilize mechanisms akin to RPE, such as instructive pathway input. Thus, theories about biologically plausible learning may be tested by connecting different organoids that act as feedforward or feedback propagation paths to mediate errors for optimal behavior.

The system 210 may also be used to implement learning in biological neural circuits (e.g., hybrid connectoid 20) by offloading some of the computation from the computational edge f into the real cortex f. The graphical representation 152 may be set up to make a larger learning loop, and patches of neurons may be recruited and trained to implement a part of the virtual edge until the edge functions are reduced to pure message-passing, basic delayed identity functions, such the computing device 212 is relieved of performing any supporting computations.

Biological mechanisms for achieving the training may be based on Hebbian learning, spike time dependent plasticity (SDTP), or instructive pathway input. Initially, the ML algorithm 400 is trained to determine how to make gradual changes. Thereafter, the mechanism may be repeated until the learning converges on the goal. This process may be first practiced in in silico simulated organoids before moving to biological organoids.

The system 210 may be used to emulate target propagation, simplified difference target propagation, and backpropagation. In particular, target propagation may be tested on the hybrid connectoids 20 by changing the activity of organoids 22 in a desired direction.

With reference to FIG. 14, illustrates counterstream networks and target propagation and shows an autoencoder 450, where for simplicity, gₗ(⋅)≈fₗ⁻¹(⋅). FIG. 14 also shows a cortical column 452 and a layer 5 pyramidal neuron 454 spanning the height of the column 452. The neuron 454 is responsible for calculating the error through compartmentalization of inputs. FIG. 14 also shows a plurality of cortical columns 452 organized in levels, mirroring the structure of the folded autoencoder 450. The autoencoder 450 is a type of the ML algorithm 400 and may be a self-supervised autoencoder neural network that is trained to copy its input to its output.

Both the feedforward networks and the self-supervised autoencoders use backpropagation. However, the brain likely does not perform backpropagation because backpropagation demands synaptic symmetry in the forward and backward paths and errors signals are signed and potentially extreme-valued (i.e., outside a spiking range of a neuron). Furthermore, gradients and neural spike activations are two completely different types of data, and neurons only propagate neural spike activations.

FIG. 14 presents a model for how a neural network may learn through target propagation without backpropagation. The biological principle is motivated by compartmental learning in pyramidal cells and predictive coding. The self-supervised autoencoder 450 is folded over on itself such that the corresponding feedforward and feedback/counterstream levels are on top of each other. As shown in panel d of FIG. 14, this allows hierarchical predictive coding by bringing the feedforward encoding and counterstream (target) representation of concept together in the same neural tissue, facilitating neural mechanisms of comparison. The network can be trained using local update rules by comparing the encoded representation to the target. Encoding (feedforward) and decoding (feedback/counterstream) are performed by functions fₗ and gₗ correspondingly. The function between two levels fₗ is similar to the one used by the feedforward networks. However, backpropagation is not necessary because the target is using single level and s is a smooth nonlinear function. When the network is trained and converges to the optimal state, gₗ(⋅)≈fₗ⁻¹(⋅), and the targets ĥₗ≈hₗ.

The error is computed by comparing the targets hi in the feedforward to the targets hi in the feedback compartments. In biology, the error could be calculated by a circuit of neurons or layer 5 neurons whose dendrites and axons measure the disparity between feedforward and feedback signals in the column. Mathematically, the error may be applied to slightly modify the functions fₗ and gₗ to produce less disparity at that layer, as in the learning mechanism in predictive coding. In biology, this would be a learning process in a neural circuit or simply the layer 5 neurons modifying connection strengths with neurons in the feedforward and feedback compartments to make the activity in these two compartments more similar.

Using the system 210, the organoids 22 can be induced to learn from structured inputs using a biologically plausible mechanism, even if perfect cortical minicolumns (i.e., sub-units of cortical columns) do not grow in the organoids, perhaps because the neural tissue self-organizes in the context of properly structured electrode stimulation into feedforward and counterstream connections. Detailed analysis of the learning behavior may then be used to distinguish between specific theoretical models of sensory processing and learning in the brain.

It will be appreciated that of the above-disclosed and other features and functions, or alternatives thereof, may be desirably combined into many other different systems or applications. Also, that various presently unforeseen or unanticipated alternatives, modifications, variations, or improvements therein may be subsequently made by those skilled in the art which are also intended to be encompassed by the following claims. Unless specifically recited in a claim, steps, or components according to claims should not be implied or imported from the specification or any other claims as to any particular order, number, position, size, shape, angle, or material.

What is claimed is:

1. An electrophysiological recording and stimulation system comprising:
an electrophysiological device coupled to neural tissue and configured to measure neural electrophysiological signals;
a computing device coupled to the electrophysiological device and configured to:
receive neural electrophysiological signals from the electrophysiological device; and
transmit neural stimulating signals to the neural tissue through the electrophysiological device based on the neural electrophysiological signals.

2. The electrophysiological recording and stimulation system according to claim 1, wherein the computing device is further configured to execute software instructions embodying a simulated neural network.

3. The electrophysiological recording and stimulation system according to claim 2, wherein the computing device is configured to provide neural communication between the simulated neural network and the neural tissue.

4. The electrophysiological recording and stimulation system according to claim 2, wherein the neural network is a reinforcement learning agent.

5. The electrophysiological recording and stimulation system according to claim 2, further comprising:
a monitor configured to display a graphical user interface including a graphical representation of the simulated neural network and the neural tissue.

6. The electrophysiological recording and stimulation system according to claim 1, wherein the electrophysiological device is a microelectrode array including a plurality of electrophysiological electrodes.

7. The electrophysiological recording and stimulation system according to claim 6, wherein the neural tissue includes at least one organoid.

8. The electrophysiological recording and stimulation system according to claim 7, further comprising a scaffold disposed on the microelectrode array.

9. The electrophysiological recording and stimulation system according to claim 8, wherein the scaffold is configured to hold a plurality of organoids in a plurality of wells.

10. The electrophysiological recording and stimulation system according to claim 9, wherein each organoid is connected to a plurality of neighboring organoids of the plurality of organoids.

11. A method for electrophysiological recording and stimulation, the method comprising:
measuring neural electrophysiological signals of neural tissue through an electrophysiological device;
receiving neural electrophysiological signals from the electrophysiological device at a computing device; and
transmitting neural stimulating signals to the neural tissue through the electrophysiological device based on instructions from the computing device.

12. The method according to claim 11, further comprising:
executing software instructions embodying a simulated neural network.

13. The method according to claim 12, further comprising:
providing neural communication between the simulated neural network and the at least one organoid through the computing device.

14. The method according to claim 12, wherein the neural network is a reinforcement learning agent.

15. The method according to claim 12, further comprising:
displaying a graphical user interface including a graphical representation of the simulated neural network and the neural tissue.

16. The method according to claim 11, wherein the electrophysiological device is a microelectrode array including a plurality of electrophysiological electrodes.

17. The method according to claim 16, wherein the neural tissue includes at least one organoid.

18. The method according to claim 17, wherein the electrophysiological device includes a scaffold disposed on the microelectrode array.

19. The method according to claim 18, wherein the scaffold is configured to hold a plurality of organoids in a plurality of wells.

20. The method according to claim 19, wherein each organoid is connected to a plurality of neighboring organoids of the plurality of organoids.
